(19) United States
(12) Patent Application Publication
(10) Pub. No.: US 2010/0179822 A1
(43) Pub. Date: Jul. 15, 2010

(54) System, Method and Device for Tracking Surgical Sponges

(76) Inventor: Soterios I. Reppas, Perry Hall, MD (US)

Correspondence Address: CAPITAL LEGAL GROUP, LLC, 1100 River Bay Road, Annapolis, MD, 21409, US

(21) Appl. No.: 12/353,876

(22) Filed: Jan. 14, 2009

(51) Int. Cl. G06Q 50/00; H04B 7/00

(52) U.S. Cl. 705/2; 340/10.1

(57) ABSTRACT

A system, method, and device for tracking surgical sponges for a medical procedure is provided. In one embodiment, the system includes an RF reader having an antenna, said antenna configured to be removably attached to a receptacle and to radiate discarded sponges comprising sponges entering the receptacle. The RF reader may be configured to receive unique identifying (ID) information from radiated sponges and provide the ID information to a wireless transmitter electrically connected to the RF reader. The system may include a handheld computer having a wireless port and configured to wirelessly receive the ID information of discarded surgical sponges from the wireless transmitter. The handheld computer may store in its memory ID information of a plurality of available surgical sponges comprising sponges available for use during the procedure and receive and store ID information of a plurality of unused surgical sponges comprising sponges that were not used during the procedure. In addition, the handheld computer may be configured to process data of the discarded surgical sponges, unused surgical sponges, and available surgical sponges to determine if any available surgical sponge is not discarded and is not unused.

### FIELD OF THE INVENTION

The present invention generally relates to systems, devices and methods for tracking medical supplies, devices and equipment, and more particularly for tracking surgical sponges and other supplies, devices and equipment that may be used during a medical procedure.

### BACKGROUND OF THE INVENTION

Medical procedures, such as surgeries, biopsies and other diagnostic and treatment procedures often require a variety of medical supplies, devices and equipment. In addition, multiple doctors, nurses and technicians may be involved. The more complex the procedure and setting, the greater the potential risk is for error. Further, as patient traffic within a setting (e.g., a given operating room) increases, and as personnel work longer shifts and perform more complex procedures, the potential for error increases.

An example of such an error is inadvertently leaving a surgical sponge within the patient's body cavity. Various tissue may be cut during a medical procedure resulting in bleeding. Surgical sponges are typically placed in the patient's body cavity to absorb the blood. After the surgical sponges become saturated with blood, they can be difficult to see in a patient's body cavity. As a result, the medical professionals sometimes overlook a sponge, leaving it in the body cavity when closing the body cavity. Leaving a surgical sponge within the patient may lead to an infection or other undesirable and dangerous reaction. Further, such error leads to increased cost, because another operation is typically required to remove the sponge. The significance of such an error is apparent by the number of times such error has been known to occur. Statistical researchers, for example, estimate that such errors occur thousands of times each year, and refer to the event as a Gossypiboma. Accordingly, there is a need for tracking surgical sponges and other supplies to provide safer, more effective medical procedures.

Further, there is a need to track the use of various devices and equipment, such as to assure that such devices and equipment are properly maintained. These and other needs may be addressed by one or more embodiments of the present invention.

### SUMMARY OF THE INVENTION

The present invention provides a system, method, and device for tracking surgical sponges for a medical procedure. In one embodiment, the system includes an RF reader having an antenna, said antenna configured to be removably attached to a receptacle and to radiate discarded sponges comprising sponges entering the receptacle. The RF reader may be configured to receive unique identifying (ID) information from radiated sponges and provide the ID information to a wireless transmitter electrically connected to the RF reader. The system may include a handheld computer having a wireless port and configured to wirelessly receive the ID information of discarded surgical sponges from the wireless transmitter. The handheld computer may store in its memory ID information of a plurality of available surgical sponges comprising sponges available for use during the procedure and receive and store ID information of a plurality of unused surgical sponges comprising sponges that were not used during the procedure. In addition, the handheld computer may be configured to process data of the discarded surgical sponges, unused surgical sponges, and available surgical sponges to determine if any available surgical sponge is not discarded and is not unused.

The invention will be better understood by reference to the following detailed description taken in conjunction with the accompanying drawings.

### BRIEF DESCRIPTION OF THE DRAWINGS

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown. In the drawings:

FIG. 1 is a block diagram of a system for tracking surgical sponges, according to an example embodiment of the present invention;

FIG. 2 is a flow chart of a method of tracking sponges according to an example embodiment of the present invention;

FIG. 3 is a flow chart of processing sponge data to track sponges according to an example embodiment of the present invention; and

FIG. 4 is a flow chart of a method for tracking surgical sponges, and other medical supplies, devices and equipment, according to an example embodiment of the present invention.

### DETAILED DESCRIPTION OF ILLUSTRATIVE EMBODIMENTS

In the following description, for purposes of explanation and not limitation, specific details are set forth, such as particular networks, devices, computers, radio frequency (RF) tags, components, techniques, data and network protocols, software products and systems, radio frequency (RF) readers, enterprise applications, operating systems, development interfaces, hardware, etc. in order to provide a thorough understanding of the present invention.

However, it will be apparent to one skilled in the art that the present invention may be practiced in other embodiments that depart from these specific details. Detailed descriptions of well-known networks, devices, computers, terminals, components, techniques, data and network protocols, radio frequency (RF) tags, radio frequency (RF) readers software products and systems, operating systems, development interfaces, and hardware are omitted so as not to obscure the description of the present invention.

According to an embodiment of the present invention, a system and method for tracking surgical sponges in real time is provided. Each sponge includes a small radio frequency identification (RFID) tag (having a unique identifying number). For example, the RFID tag may be embedded in the sponge and/or otherwise attached to the sponge, such as with a hand tag located along a string attached to the sponge. Sponges may be scanned prior to use and after use to track and count the sponges. For example, when preparing an operating room for a medical procedure (and prior to the procedure) each sponge may be scanned. Each sponge may have a unique identifying number, allowing specific sponges to be tracked and for counting the number of sponges and the number of each type of sponge.

After a sponge is used, it is discarded. Because the sponges soak up blood and other bodily fluids, the used sponges are biohazardous waste. In some embodiments, a hazardous waste bin may have an attached loop antenna through which sponges pass when being discarded into the waste bin.

An RF reader may be electrically connected to the antenna to read the RFID tag ID of each sponge discarded. In turn, the RFID reader provides the ID data to a wireless transceiver that wirelessly transmits the RFID tag ID (in encrypted form) to a computer (e.g., a local handheld Bluetooth device, laptop, or network terminal). In an example embodiment, software resident on the computer executes program code to identify the number of sponges provided for a procedure, the number of sponges remaining unused after the procedure, and the number of sponges discarded during (and after) the procedure. The computer then determines if all sponges are accounted for or if a discrepancy exists. Further, an alarm or other notification may be generated when there is a discrepancy. Of course, variations of the method may be implemented, such as a method in which only sponges that are opened for imminent use and sponges that are discarded are scanned and the counts (or ID data) compared.

The system and method also may be implemented to track other medical supplies, devices and equipment used during a medical procedure.

### Tracking System:

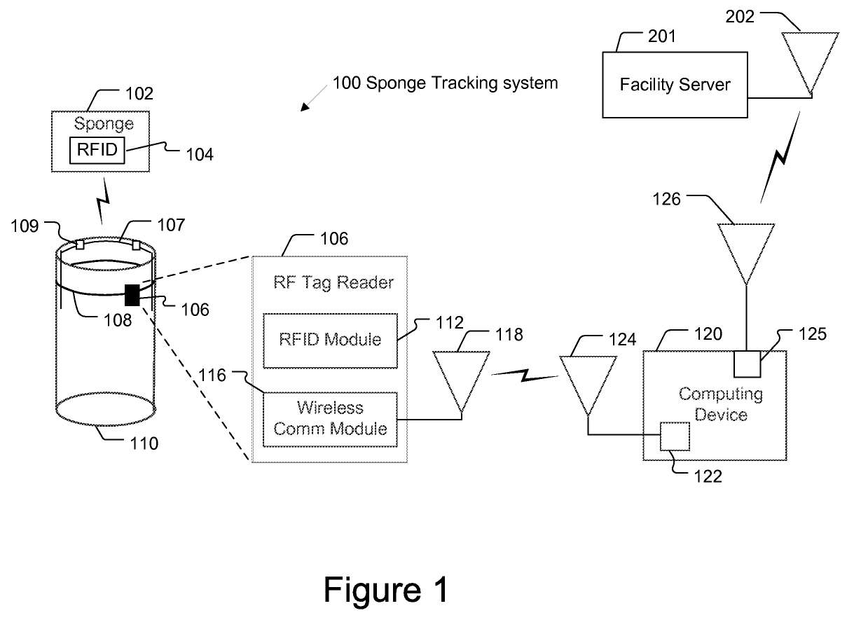

FIG. 1 depicts a system 100 for tracking surgical sponges 102, according to an example embodiment of the present invention. The tracking system 100 includes medically non-reactive RFID tags 104 embedded and/or otherwise attached to one or more surgical sponges 102. An RFID tag 104 serves as a transponder which responds to radio frequency irradiation.

As discussed, each RFID tag has an associated unique ID (e.g., a number). Some RF ID tags can be read from several meters away and beyond the line of sight of the reader. Other tags are less robust. Most RFID tags contain at least two parts. A first part is an integrated circuit for storing and processing information, for modulating and demodulating a radio-frequency (RF) signal, and for other specialized functions. The second part is an antenna for receiving and transmitting the RF signal. It is anticipated that RFID technology will soon omit the chip component and instead allow the RFID tags to be printed directly onto the surgical sponge or other asset being tracked. In this example embodiment, a passive RFID tag 104 responds to a radio frequency signal of sufficient power for the tag's antenna to detect the signal and power the integrated circuit to encode the ID information. In effect the RFID tag 104 is a transponder powered by the incoming signal to transmit the RFID tag ID information in response to a specific incoming radio frequency signal. In another embodiment, the RF ID tags are active tags (e.g., include a power source such as a battery).

An example of an RFID tag 104 suitable for use with a surgical sponge for some embodiments of the present invention may be a Gen 2, 96-bit, passive RFID tag. A read only read or a read/write tag may be employed. The RFID tag 104 may be fixed, tethered or otherwise attached to the asset to be tracked. For example, an RFID tag 104 may be woven into a surgical sponge 102. In some embodiments, the sponge may be tethered to the sponge and encased in a plastic housing that includes a recessed ridge for receiving forceps for extraction or a recessed thumbprint for receiving a person's thumb to facilitate manual extraction. In some embodiments the sponge 102 may include a hole near an edge for a tethered cord to be connected (in which such an RFID tag 104 may be located).

The system 100 also includes an RFID tag reader 106 including an RF module 112 electrically connected to an antenna 108. The RFID module 112 of the RFID tag reader 106 transmits a radio frequency signal via the antenna 108. In various embodiments, the radio frequency signal may be generated automatically and transmitted periodically or aperiodically (or continuously). The radio frequency signals radiate radio RFID tags 104 located within the radiating field of the antenna 108 to allow the RFID tags 104 to provide a response. In a specific embodiment the radio frequency signal may be an Ultra High Frequency (UHF) signal within the range of 902 through 928 MHz.

The antenna 108 may be a loop antenna or loop/dipole antenna oriented to have peak intensity in a specific plane. In various embodiments the antenna 108 may be read in near field or far field modes. For example, the antenna 108 may be located around the circumference of an opening of the waste bin 110 which receives used discarded sponges. The loop antenna 108 may be positioned, for example, by attaching an apron 107 with hooks 109 over the opening of the discard bin 110. The apron may be angled so when a sponge is discarded, the sponge 104 passes through the loop opening and drops into the bin 110. The apron 107 may be made of disposable material so that it could be discarded after each procedure. In this example, the antenna's peak intensity may occur in the plane of the loop so that a sponge's RFID tag 104 is radiated when passing through the waste bin's opening. In some embodiments, the antenna 108 may be suspended below the opening, with energy propagating upward to radiate the entire opening of the waste bin 110.

In another embodiment, the antenna 108 may be located along a wall of the discard bin 110 and have a peak intensity perpendicular to the plane of the loop. The antenna may radiate an entire portion of the waste bin 110, such as the area near the bin's opening.

Although not specifically depicted, the RF tag reader 106 also includes a power supply and filtering circuitry for receiving power from a conventional wall socket. In another embodiment, the RF tag reader 106 may be battery powered. The RF module 112 may include a transceiver for transmitting and receiving RF signals. For example, the RF module 112 may generate a RF beacon signal or other RF signal output to be radiated by the antenna 108. As discussed, the output signal is a radio frequency signal transmitted at sufficient power to excite the RFID tags 104 that may pass or otherwise be located within a desired radiating field of the antenna 108.

The RF module 112 also may receive RFID signals containing ID information from the RFID tags 104 from the antenna 108. In particular, the RFID module's RF output signal causes the near field antenna 108 to radiate an output field. An RFID tag passing through such field may be powered by such radiation to generate an RFID signal with ID information of the associated sponge. The RFID signal may repeat while the RFID tag 104 remains exposed to the radiation from antenna 108. The ID information of the RFID signal may be used to determine the sponge which was discarded into the waste bin 110.

The received ID information may then be provided to the wireless communication module 116 by the RF module 112. In some embodiments, the RF module 112 may decode and/or reformat the ID data carried in the RFID signal. The wireless communication module 116 receives the ID information from the RFID module 112 and packages (e.g., formats) the data for wireless transmission. An antenna 118 may be electrically connected to the wireless communication module 116. Various wireless protocols may be implemented, such as Bluetooth, wireless USB, ultra wideband, and the various wireless local area network (WLAN) protocols defined by the IEEE 802.11 family of specifications, (e.g., 802.11, 802.11a, 802.11b, 802.11g, and 802.11n).

The system 100 for tracking surgical sponges 102 also may include a computing device 120, such as a personal computer, laptop, handheld device, cart on wheels, or network terminal. The ID information from the radiated RFID tags 104 may be transmitted via a wireless Bluetooth communication to the computing device 120 where the data is stored and processed. Thus, the computing device 120 may include a processor, memory, and a wireless communication module 122 having a short range transceiver and antenna 124 for communicating with the RF tag reader. In addition, the computing device 120 may include a second transceiver 125 and antenna 126 for communications (e.g., Bluetooth, IEEE 80.11a/b/g/n (Wifi)) with a remote computer system 201 such as a server configured manage inventory, billing, maintenance and other processes. A single computing device 120 may be configured to operate with multiple RF Tag readers 106 concurrently or separately.

In some embodiments, the computing device 120 may store software for executable to control the process of tracking surgical sponges. Reports, alarms, and other outputs may be generated as part of the tracking process. The data, and the process outputs also may be sent to another computing system for archiving the data and the results. The computing device 120 or other computing system also may pass data to an electronic patient record system.

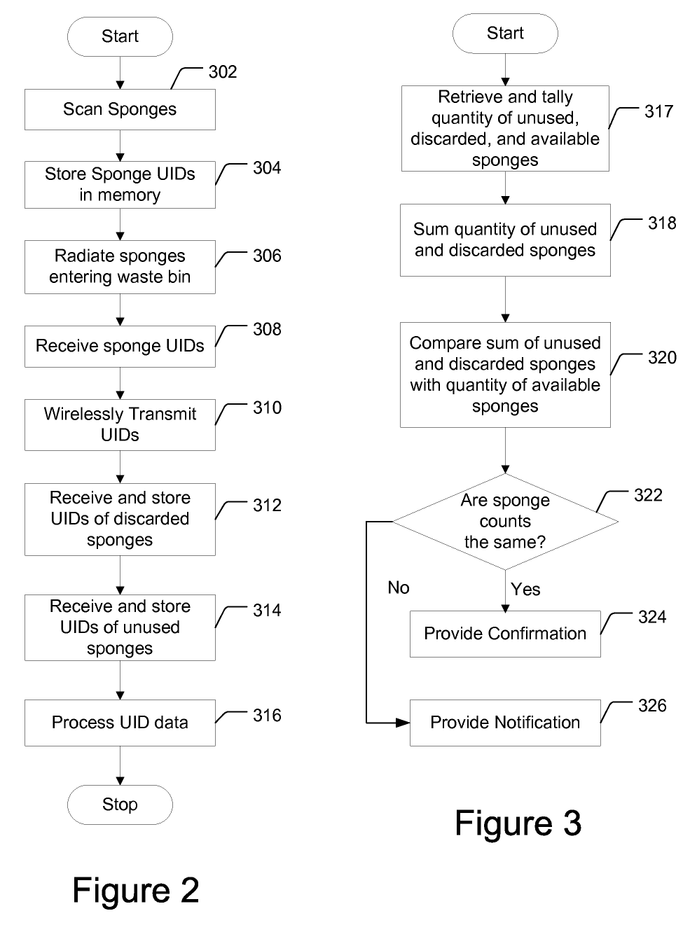

FIG. 2 depicts a process for tracking surgical sponges in accordance with an example embodiment of the present invention. At step 302, all of the sponges provided to the operating room are scanned prior to the beginning of the medical procedure. These sponges are referred to herein as the available sponges (because they are available for use during the procedure). The unique IDs (UIDs) of the available sponges (and total quantity of available sponges) are stored in memory of the computing system 120. The sponges may be scanned by any suitable RFID scanning device such as a handheld scanner or a scanning device that is electrically (or wirelessly) connected to the computing device 120. Alternately, the sponges may be placed through the hoop antenna 180 of the RFID tag reader 106 (prior to the antenna being fasted to the waste bin 110) in which case the computing device 120 receives the UIDs via a wireless transmission from the RF tag reader 106. At step 304, the UIDs of the sponges available for use during the procedure are stored in memory of the computing device 120. Medical personnel may scan additional sponges to add (e.g., to the total count of available sponges) if, during the medical procedure, additional sponges are needed.

At step 306, the sponges entering the waste bin 110 are radiated by the RF tag reader 106. In instances, the reader may concurrently radiate multiple sponges entering the receptacle concurrently and receive the UIDs. At step 308, in response to the radiating, the RF tag reader receives the UIDs of the sponges entering the waste bin 110. At step 310, the received UIDs are wirelessly transmitted (e.g., via a Bluetooth transmission) to the computing device 120 for storage and processing. At step 312, the computing device 120 receives and stores the UIDs of the discarded sponges. As will be evident to those skilled in the art, steps 306-312 (or alternately only steps 306 and 308) may occur each time a sponge enters the waste bin 110.

When the procedure is complete (and before the patient's body cavity is closed), the unused sponges may be scanned. At step 312, the computing device 120 receives and stores the UIDs of the unused sponges. For example, when the medical procedure is near completion and all of the sponges are believed to be removed from the patient and discarded, a medical professional may provide a user input to indicate the scanning of unused sponges. Such scanning may be performed via any suitable method as described above.

When all of the unused sponges have been scanned, the computing device 120 may then process the UID data to identify any discrepancy in the number of available sponges with the number of sponges unused and discarded. The processing may be done in response to a user input to process the UID data. The processing may result in a report that indicates whether any (and how many) unused sponges where not discarded or that all used sponges were discarded.

FIG. 3 depicts an example method for processing of the UID data (step 316) to identify any discrepancy in the number of available sponges with the number of sponges unused and discarded. At step 317, the UIDs of the unused, discarded and available sponges (i.e., sponges available for use during the medical procedure) are retrieved from memory (based on the UID data) and tallied to provide a quantity for each. At step 318, the number of unused sponges (derived from step 314) is added to the number of discarded sponges (derived from step 312) to provide a post procedure sponge count.

At step 320, the post procedure sponge count is compared to the number of available sponges (i.e., the number of sponges available prior to the procedure) derived from step 304. At step 322, the process determines whether the sponge counts (i.e., the available sponges and the post procedure sponge count) are the same. If the sponge counts are the same, the process branches to step 324 and provides a notification confirming that all sponges are accounted for (and none remains in the patient). Upon receiving this confirmation notification, the medical professionals will be confident knowing that no sponges remain in the patient's body cavity and may close the patient's body cavity.

If the sponge counts are not the same (i.e., a discrepancy exists), the process branches to step 326 and provides an alert notification that not all sponges are accounted for. This notification may indicate the number of sponges that were made of available for use (available sponges) and that were neither discarded nor remain unused. Upon receiving this notification, the medical professionals may then seek to locate the one or more sponges that are missing. For example, the medical professional may scan the patient to see if the one or more patients remain in the patient's body cavity. Upon finding the missing sponges, they may be scanned (either via being discarded or via scanning) and the processing of the UID data re-initialized to ensure that no discrepancy exists between the sponges that were (1) made of available for use (available sponges) and (2) those that were discarded or remain unused.

In another embodiment, the method of determining a discrepancy in the number of available sponges with the number of sponges unused and discarded comprises comparing the UIDs of the discarded surgical sponges and the unused surgical sponges with the UIDs of the available surgical sponges to determine if any available surgical sponge is not discarded and is not unused. In other words, instead of tallying the quantity of discarded, unused, and available sponges, the computing system compares the UID (e.g., the ID numbers) of each unused and discarded sponge with the UIDs of the available sponges. For example, the computing device 120 may create a database table that includes the UIDs of the available sponges forming part of each record. As each sponge is later scanned (as a discarded or unused sponge) a flag is inserted into a field of the corresponding UID record. Upon completion of the procedure and scanning of unused sponges, any record in the table that does not include the flag identifies a missing sponge that may remain in the patient. The number of records without a flag may be tallied to provide the quantity of missing sponges.

The present invention also be implemented to track a variety of types of assets (medical supplies, devices and equipment) used or made available for use for medical procedures such as peel packs (surgical instruments), items from materials management (e.g., sponges, sutures, syringes, etc.), electronic equipment (e.g., EKG's, IV pumps, and respirators) from equipment/fixed asset management, consumables such (e.g., drugs, infusion therapy, blood, and saline) from pharmacies and blood banks, and various other types of assets. Data of these assets may be stored in a remote computer system 201 located anywhere in the facility or elsewhere. For instance, all medical equipment can be logged into a fixed asset system (e.g., equipment) where a complete historical profile and maintenance history is created and maintained. If an IV pump is used in an operation the attending nurse can (in real time) pull up the disposition and maintenance history of that particular piece of equipment. Potential problems may be uncovered before they happen in a critical stage of the operation. If the IV pump is past due on it's a maintenance check, the nurse can get a new IV pump.

The assets may be scanned by one or more RF tag readers 106 in the procedure room. In some embodiments, the procedure record (that includes data of the assets used during a procedure) may be processed to provide a billing entry in the facility's billing system, a chart note in a patient's electronic medical record, a scheduling entry in a scheduling system noting the end of the procedure, a maintenance entry indicating maintenance of a piece of equipment is due, an inventory entry decrementing the inventory of one or more items, and/or another entry in any of various information management systems for the patient, surgical team, or facility. In embodiments, bar codes may be scanned to track assets having bar codes attached thereto (in addition to or alternate to using RFID tracking technology).

To track a variety of assets various data may be entered prior to the medical procedure. For example, the room number for the procedure may be entered into the computing device 120. In addition, an employee ID or other data corresponding to a given person or team of people may be entered. A patient ID or other data (e.g., name) indicating a particular person who is to undergo the procedure is entered. A procedure code, such as a CPT code (i.e., current procedural terminology code) also may be entered. In some embodiments a current time or date may be entered or stored. In various embodiments some or all of these steps may be performed in any order. In some embodiments, such data is entered at the computing device 120 or other computer (e.g., that is connected to the remote computer system). For example, such data may already be in a hospital's information management system and be automatically associated with a patient, doctor, technician or room based upon prior scheduling data entries. In such example, these steps need not be performed at the computing device 120, but at other computing systems 201 that may or may not make such data available to the computing device 120.

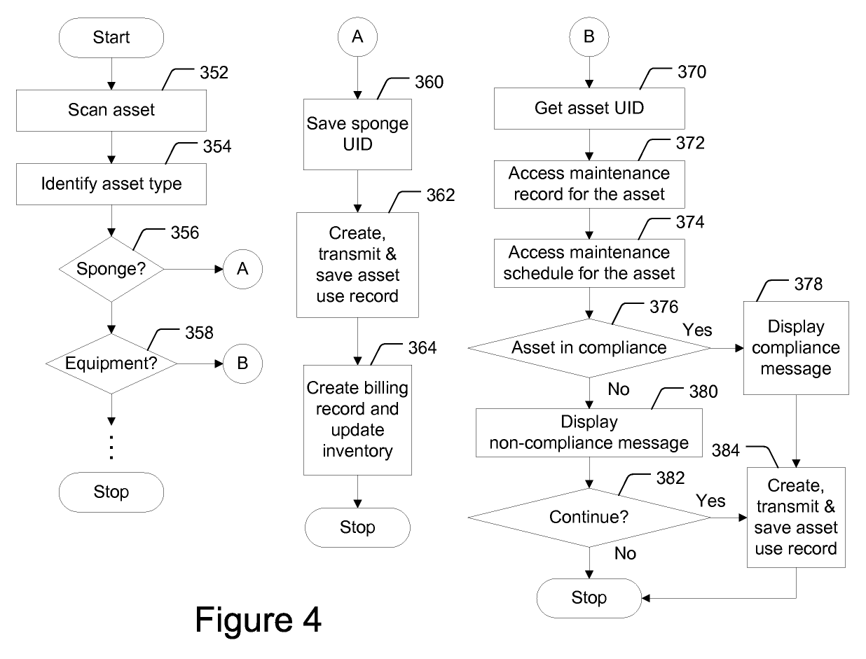

FIG. 4 depicts a method 350 for processing data provided by the scanning of an asset with an RF tag reader (such as RF tag reader 106 or other scanning device such as a bar code reader). As discussed, such scanning may be performed on various assets at various times before, during and after a procedure. At step 352, a given asset is scanned. The asset may comprise any asset that the facility wishes to track such as a material (e.g., a sponge), a consumable (e.g., a medication), equipment (e.g., an EKG machine or IV pump), etc. Thus, in this example embodiment there are a plurality of types of assets that can be scanned. At step 354 the UID from the asset is read and the type of asset is determined such as by the computing device 120 or the remote computer 201. For example, the asset type may be encoded on the RFID tag 104, or may be retrieved from a database that correlates various UID to asset types. The type of asset scanned may determine (in some instances) the process to be next performed. For example, at step 356 the asset type may be determined to be a surgical sponge. If so, then steps 360-362 may be performed. At step 358 the asset type may be determined to be a piece of equipment and steps 370-384 may be performed. Various asset types may be detected to determine a set of processes to be performed for such asset or asset type.

If the asset type is a surgical sponge 102, at step 360 the sponge's UID is stored. At step 362 an asset use record may be stored, transmitted and saved. For example, in embodiments where the computing device 120 includes procedure data, an asset use record may be created, transmitted and stored. In a specific example, the UID of the sponge, along with a patient id, room number, employee id and procedure code may be stored as an asset use record. In some embodiments a time and date stamp also may be included in the asset use. Such record may be sent by the computing device 120 to a remote computer system 201 (e.g., wirelessly via Bluetooth or Wifi or via wired communication). Such remote computer system 201 (discussed above) may comprise a central computer system of the medical facility (or one or more facilities) and be located in the same structure (e.g., in a “computer room”) or remote therefrom (at a data center in which case the communication may traverse the internet).

The remote computer system 201 may save the data and process the data to create a billing record and update an inventory management system at step 364. For example, the remote computer system may create billing records to bill the patient for each surgical sponge discarded (and not bill the patient for sponges not used) as well as other consumable used during a procedure. In some embodiments, the type of sponge may also be determined from the UID information. This may be used to more accurately bill the patient (e.g., different types of sponges may have different costs) as well as to track the remaining inventory of each type of surgical sponge.

As discussed, steps 370-384 may be performed in response to a scan of an equipment asset type such as an EKG or IV pump. At step 370, the asset's UID is received from the scanning. The computing device 120 (or the remote computer system 201) may then determine whether that particular piece of equipment has been maintained according to a prescribed schedule. Specifically, at step 372 the maintenance record for that particular piece of equipment may be retrieved and at step 374 the maintenance schedule for that type of asset (or asset sub-class) is retrieved. At step 376, the process determines whether the retrieved asset maintenance activity is up to date or otherwise in compliance with the retrieved maintenance schedule. Various other processes also may be performed depending on the needs of the facility and type of asset.

If the asset is in compliance with prescribed test criteria, (e.g., the EKG has been maintained according to a maintenance schedule), then a message may be displayed at step 378 at the RF reader 106 indicating such compliance (or no message provided). A record of the asset's use may be generated and saved at step 384. Similarly, if the asset is not in compliance, then a notification may be displayed at step 380 on the computing device 120 that the asset is not in compliance with the maintenance schedule. In a specific example, the UID, along with the patient ID, room number, employee ID and procedure code may be stored as the asset use record. In some embodiments a time and date stamp also may be included in the asset use record. Such record may be sent by the computing device 120 to the remote computer system 201, as previously described. Such remote computer system 201 may save the record and/or process the record to perform billing, patient medical record generation or other accounting, schedule maintenance, or maintaining operation.

For the case where the asset is not in compliance with test criteria, the operator may be asked whether or not to continue use of such asset. For example, the operator may choose to ignore the alert message and proceed by using the piece of equipment for the medical procedure. In such case, an asset use record may be generated at step 384. Alternatively, the operator may choose not to use the piece of equipment. In such case the asset use record need not be generated. Alternatively, in some embodiments an alternative asset use record may be generated such as to record that the piece of equipment was found not to comply with test criteria. Typically, the operator would find and scan a substitute piece of equipment which complies with the test criteria. In some embodiments, some (e.g., equipment, peel packs, ancillaries, etc.) or all of the assets may be tracked using alternate technology such as, for example, bar codes to implement the embodiments described herein.

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A method of using a system to track surgical sponges for a medical procedure, comprising:
storing in a memory of a computer a multitude of identifiers (IDs) with each ID corresponding to a different one of a multitude of available surgical sponges that are available for use for the medical procedure;
radiating a plurality of discarded surgical sponges entering a waste receptacle;
receiving, from said radiating, a plurality of IDs, each received ID corresponding to one the plurality of sponges entering the waste receptacle;
wirelessly transmitting each of the plurality of received IDs to the computer;
receiving the plurality of IDs at the computer;
receiving at the computer one or more of the multitude of IDs at completion of the medical procedure, wherein the one or more IDs correspond to surgical sponges not used during the procedure; and
determining whether any available surgical sponge is not discarded and is not unused based on:
(a) the multitude of IDs corresponding to the multitude of surgical sponges available for use for the medical procedure;
(b) the plurality of IDs corresponding to the plurality of sponges entering the waste receptacle; and
(c) the one or more IDs correspond to surgical sponges not used during the procedure.

2. The method according to claim 1, wherein said determining comprises:
determining a quantity of available sponges by tallying the quantity of the multitude of IDs corresponding to the multitude of surgical sponges available for use for the medical procedure;
determining a quantity of discarded sponges by tallying the quantity of the plurality of IDs corresponding to the plurality of sponges entering the waste receptacle;
determining a quantity of unused sponges by tallying the quantity of the one or more IDs corresponding to surgical sponges not used during the medical procedure; and
determining a sum of the quantity of discarded sponges and the quantity of unused sponges; and
comparing the sum with the quantity of available sponges.

3. The method according to claim 2, further comprising generating a notification if there is a discrepancy between the sum and the quantity of available sponges.

4. The method according to claim 1, further comprising storing billing data in a storage device and wherein said billing data is based on a quantity of the plurality of IDs corresponding to the plurality of sponges entering the waste receptacle.

5. The method according to claim 4, wherein the billing data is further based on the type of each sponge discarded.

6. The method according to claim 1, wherein said determining comprises comparing the IDs of the sponges entering the waste receptacle and the IDs of sponges not used with the IDs of the sponges available for use for the medical procedure to determine if any surgical sponge available for use is not discarded and is not unused.

7. The method according to claim 6, further comprising generating a notification if any surgical sponge available for use is not discarded and is not unused.

8. The method according to claim 6, further comprising storing billing data in a storage device and wherein said billing data is based on a quantity of the surgical sponges discarded.

9. The method according to claim 6, further comprising storing inventory data in a storage device and wherein inventory data is based on a quantity of the surgical sponges discarded.

10. The method according to claim 1, further comprising providing a plurality of surgical sponges for use during the medical procedure, each having an associated radio frequency identification (RFID) tag tethered to the sponge.

11. The method according to claim 10, wherein the RFID tag is disposed in a housing having a depression to receive a human digit.

12. The method according to claim 1, further comprising providing a plurality of surgical sponges, each having an associated radio frequency identification (RFID) tag woven into the sponge.

13. A system for tracking surgical sponges for a medical procedure in which used sponges are discarded into a receptacle, comprising:
an RF reader having an antenna, said antenna configured to be removably attached to the receptacle and to radiate discarded sponges entering the receptacle;
said RF reader configured to receive identifying (ID) information from radiated discarded sponges;
a wireless transmitter electrically connected to said RF reader to receive from said RF reader the ID information of discarded sponges;
a portable computer having a wireless port and configured to wirelessly receive the ID information of discarded sponges from said wireless transmitter;
said portable computer storing in memory ID information of a plurality of available surgical sponges comprising sponges available for use for the procedure;
wherein said portable computer is configured to receive and store ID information of a plurality of unused surgical sponges comprising sponges available for use for the procedure and that were not used during the procedure; and
wherein said computer is responsive to an input to process data of the discarded surgical sponges, unused surgical sponges, and available surgical sponges to determine if any available surgical sponge is not discarded and is not unused.

14. The system of claim 13, further comprising a plurality of surgical sponges, each having an associated radio frequency identification (RFID) tag.

15. The system of claim 14, wherein said RFID tag of each sponge is tethered to the sponge.

16. The system to claim 15, wherein said RFID tag is disposed in a housing having a depression that is shaped to receive a human thumb.

17. The system of claim 14, wherein said RFID tag of each sponge is woven into the sponge.

18. The system of claim 13, wherein said wireless transmitter comprises a Bluetooth transceiver.

19. The system of claim 13, wherein said portable computer comprises a handheld computing device having a Bluetooth transceiver.

20. A system for tracking surgical sponges for a medical procedure in which used sponges are discarded into a waste receptacle, comprising:
an RF reader having an antenna enclosed, at least in part, in a disposable apron;
wherein said antenna is configured to be removably attached to the receptacle and to radiate discarded sponges entering the receptacle;
wherein said apron is configured to be removably attached to said antenna;
wherein said RF reader is configured to receive identifying (ID) information from radiated sponges comprising;
a wireless transceiver electrically connected to said RF reader to receive from said RF reader the ID information of discarded sponges; and
wherein said wireless transceiver is configured to wirelessly transmit ID information of discarded sponges.

21. The system according to claim 20, further comprising:
a computer having a wireless port and configured to wirelessly receive the ID information of discarded sponges from said wireless transceiver;
said computer storing in memory ID information of a plurality of available surgical sponges comprising sponges available for use for the procedure;
wherein said computer is configured to receive and store ID information of a plurality of unused surgical sponges comprising sponges available for use for the procedure and that were not used during the procedure; and
wherein said computer is responsive to an input to process data of the discarded surgical sponges, unused surgical sponges, and available surgical sponges to determine if any available surgical sponge is not discarded and is not unused.
